(19) United States
(12) Patent Application Publication
(10) Pub. No.: US 2003/0228352 A1
(43) Pub. Date: Dec. 11, 2003

(54) Cleansing articles for skin or hair

(75) Inventors: Erik John Hasenoehrl, Loveland, OH (US); Matthew Justin Paquette, Medina, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/453,169

(22) Filed: Jun. 3, 2003

Related U.S. Application Data
(60) Provisional application No. 60/387,297, filed on Jun. 7, 2002.

(51) Int. Cl. A61K 9/70

(52) U.S. Cl. 424/443

(57) ABSTRACT

The present invention relates to disposable, personal cleansing articles useful for cleansing the skin or hair. These articles are used by the consumer by wetting the dry article with water and then rubbing the article against the skin or hair. The article comprises a water insoluble substrate having a cleansing surface and comprising latex-coated cellulosic fibers, and a lathering surfactant releasably associated with the substrate. Preferably, the articles of the present invention further comprise a conditioning component.

### CROSS REFERENCE TO RELATED APPLICATION

This application claims the benefit of U.S. Provisional Application No. 60/387,297, filed Jun. 7, 2002.

### TECHNICAL FIELD

The present invention relates to disposable personal cleansing articles useful for cleansing, and optionally conditioning, the skin or hair, and more particularly to a disposable, cleansing article comprising a substrate and a lathering surfactant component. The substrate may include one or more layers, at least one of which comprises latex coated cellulosic fibers. The substrate may optionally include a plurality of apertures. These cleansing articles are used by the consumer by wetting the article with water and by thereafter forming a lather by rubbing the article against itself and/or against skin or hair.

### BACKGROUND OF THE INVENTION

Personal cleansing products have traditionally been marketed in a variety of forms such as bar soaps, creams, lotions, and gels. These cleansing formulations have attempted to satisfy a number of criteria to be acceptable to consumers. These criteria include cleansing effectiveness, skin feel, mildness to skin, hair, and ocular mucosae, and lather volume. Ideal personal cleansers should gently cleanse the skin or hair, cause little or no irritation, and not leave the skin or hair overly dry after frequent use. Personal cleansing products are frequently used with, or marketed in the form of, articles that employ a substrate or other implement that carries a cleansing material or is used to deliver a cleansing material to the skin or hair.

Traditional forms of personal cleansing products and articles may be very useful for providing efficacious cleansing and lathering. Such conventional products and articles, however are less suitable for also simultaneously providing other desirable effects such as delivering a skin or hair conditioning benefit. One solution to this problem is to use separate cleansing and conditioning products or articles. However, this is not always convenient or practical, and many consumers would prefer to use a single article which can both cleanse and condition the skin or hair. In a typical cleansing composition or product, the conditioning ingredients are difficult to formulate because many conditioners are incompatible with the surfactants, resulting in an undesirable non-homogenous mixture. To obtain a homogeneous mixture with conditioning ingredients, and to prevent the loss of conditioning ingredients before deposition, additional ingredients, e.g. emulsifiers, thickeners, and gellants are often added to suspend the conditioning ingredients within a surfactant mixture. This results in an aesthetically pleasing homogenous mixture, but often results in poor deposition of conditioning ingredients onto skin or hair because the conditioners are emulsified and not efficiently released during cleansing. Also, many conditioning agents have the disadvantage of suppressing lather generation. Lather suppression is a problem because many consumers seek cleansing articles that provide a rich, creamy, and generous lather.

Therefore, it is seen that conventional cleansing products and articles which attempt to combine surfactants and other materials such as conditioning ingredients suffer from disadvantages inherently resulting from the incompatibilities of surfactants and conditioners. A need clearly exists to develop cleansing systems which provide effective cleansing, effective lathering and yet can also, if desired, consistently provide other benefits such as sufficient conditioning in a single article.

It is also highly desirable to deliver cleansing and preferably conditioning benefits from a disposable, single use article. Disposable articles are convenient because they obviate the need to carry cumbersome bottles, bars, jars, tubes, and other forms of both cleansing and conditioning articles. Disposable articles are also a more sanitary alternative to the use of a sponge, washcloth, or other cleansing implement intended for multiple reuse, because such implements develop bacterial growth, unpleasant odors, and other undesirable characteristics related to repeated use.

### SUMMARY OF THE INVENTION

The present invention relates to disposable, single use personal cleansing articles especially useful for cleansing facial skin. Each such article comprises a) a water-insoluble, latex-coated cellulosic substrate having at least one cleansing surface; and b) from about 0.5% to 250% by weight of the substrate of a lathering surfactant which is releasably associated with the substrate.

In accordance with particular embodiments of the invention, the cleansing surface of the substrate may contain a plurality of apertures which range in size from about 0.5 mm to 5 mm in diameter. These apertures are located within the cleansing surface of the substrate at a frequency of from about 0.5-12 apertures per linear centimeter. The cleansing articles of this invention may comprise multiple layers. Apertures, when present, may be utilized in one or more of the layers. In accordance with certain manifestations of this invention, the cleansing articles utilize a two-ply substrate wherein one or both plies are apertured. Also in accordance with some embodiments at least one of the plies of the substrate is wet extensible and the second ply is less wet extensible than the first ply. Some of the cleansing articles described herein contain one or more water-soluble or water-insoluble conditioning agents in addition to the lathering surfactant component.

The present invention also relates to methods for manufacturing cleansing articles of the configuration described herein. Also, the present invention provides methods for cleansing, and optionally conditioning, the skin or hair using the articles described herein.

### BRIEF DESCRIPTION OF THE DRAWINGS

FIG. 1 is a plan view illustration of one embodiment of a cleansing article of the present invention, the article including an extensible, apertured top layer and a less extensible base layer, with the top layer shown facing the viewer, and with a portion of the top layer shown cut away to show a continuous network of generally parallel sets of intersecting lines of adhesive which serve to bond the top layer to the base layer, the bonded region defining generally diamond-shaped unbonded regions.

FIG. 2A is a cross-sectional illustration of the cleansing article of the present invention taken along the direction indicated by line 2-2 in FIG. 1, and showing the article prior to wetting of the apertured top layer.

FIG. 2B is a cross-sectional illustration taken along the direction indicated by line 2-2 in FIG. 1, and showing the article after wetting of the apertured top layer.

### DETAILED DESCRIPTION OF THE INVENTION

The essential elements of cleansing articles of the present invention, i.e., the latex coated cellulosic substrate and the lathering surfactant, as well as a wide variety of optional elements, are described in detail as follows. All percentages and ratios used herein, unless otherwise indicated, are by weight and all measurements made are at 25° C., unless otherwise designated. The invention hereof can comprise, consist of, or consist essentially of, the essential as well as optional ingredients and components described therein.

By a “lathering surfactant” is meant a surfactant, which when combined with water and mechanically agitated generates a foam or lather. Preferably, these surfactants should be mild, which means that these surfactants provide sufficient cleansing or detersive benefits but do not overly dry the skin or hair (e.g., removing too much natural oil and/or moisture), and yet meet the lathering criteria described above.

The term “lathering product” or “lathering article,” as used herein, means that the product or article contains enough of the surfactants described herein that it can generate ∃ 30 ml of Lather Volume, as described herein in the Lather Volume Test. These Lather Volume measurements are conducted with a medium hardness water (8-10 grains per gallon) at 35° C. (95° F.).

The terms “disposable” or “single use,” are used herein in their ordinary sense to mean an article that is disposed or discarded after a typical usage event.

The term “conditioning component,” as used herein, means a combination of the conditioning agents.

The term “water-activated,” as used herein, means that the present invention is presented to the consumer in a form to be used after it is wetted with water. It is found that these articles produce a lather or are “activated” by contacting them with water and then further subjecting the article to mechanical forces, such as rubbing.

The term “substantially dry,” as used herein, means that prior to use the article is substantially free of water and generally feels dry to the touch. Thus, the articles of the present invention will generally comprise less than about 20% by weight of water, preferably less than about 10% by weight of water, and more preferably less than about 5% by weight of water, the forgoing measured in a dry environment, e.g., low humidity. One of ordinary skill in the art would recognize that the water content of an article such as in the present invention can vary with the relative humidity of the environment.

The term “mild” as used herein in reference to the lathering surfactants and articles of the present invention means that the articles of the present invention demonstrate skin mildness comparable to a mild alkyl glyceryl ether sulfonate (AGS) surfactant based synthetic bar, i.e., synbar. Methods for measuring mildness, or inversely the irritancy, of surfactant containing articles, are based on a skin barrier destruction test. In this test, the milder the surfactant, the lesser the skin barrier is destroyed. Skin barrier destruction is measured by the relative amount of radio-labeled (tritium labeled) water (3H—H₂O) which passes from the test solution through the skin epidermis into the physiological buffer contained in the diffusate chamber. This test is described by T. J. Franz in the J. Invest. Dermatol., 1975, 64, pp. 190-195; and in U.S. Pat. No. 4,673,525, to Small et al., issued Jun. 16, 1987. Other testing methodologies for determining surfactant mildness well known to one skilled in the art can also be used.

The personal cleansing articles of the present invention comprise the following essential components: (A) a water-insoluble substrate, wherein at least a portion of said substrate contains cellulosic fibers bound together with a latex binder, and (B) at least one lathering surfactant added onto or impregnated into the substrate. The articles of the present invention can further optionally comprise a conditioning component added onto or impregnated into the substrate.

Water Insoluble Substrate

The products of the present invention comprise a water insoluble substrate having at least one cleansing surface. By “water insoluble” is meant that the substrate does not dissolve in or readily break apart upon immersion in water. The water insoluble substrate is the implement or vehicle for delivering the lathering surfactant and optionally the conditioning component of the present invention to the skin or hair to be cleansed and conditioned. Without being limited by theory, it is believed that the substrate, by providing mechanical forces and agitation provides a lather generating effect and also aids in the deposition of the conditioning component.

A wide variety of materials can be used as the substrate. The following nonlimiting characteristics are desirable: (i) sufficient wet strength for use, (ii) sufficient abrasivity, (iii) sufficient loft and porosity, (iv) sufficient thickness, and (v) appropriate size.

In accordance with the present invention, at least a portion of the substrate comprises cellulosic fibers bound together with a latex binder. The substrate can have a basis weight of between about 25 to about 150 grams per square meter. In accordance with a particular embodiment, the basis weight of the substrate is between about 40 to about 100 grams per square meter, and in a more particular embodiment, the basis weight of the substrate is about 45 to about 75 grams per square meter.

The latex coating on the cellulosic fibers provides strength, softness, hand, etc. The substrate of the present invention feels like a non-woven even though it is cellulose or paper based. The fibers can be coated with the latex coating by any means known to those skilled in the art: for example, by spraying, jet printing, splashing, dipping, soaking, or coating (e.g., slot coating, gravure, etc.). Nonlimiting examples of latices useful herein include those selected from the group consisting of acrylic, polyvinyl alcohol (PVA), vinyl acrylic, acrylic multipolymer, ethylene vinyl acetate (EVA) and copolymers and mixtures thereof. Specific examples of commercially available latices useful herein include Perstorp Clariant Appretan TE 2540 S (copolymer of vinylacetate and ethylene), H. B. Fuller PD8161 (PVA), H. B. Fuller PD0495 (vinyl acrylic) and H. B. Fuller PD8169 (acrylic multipolymer). The latex coating can be applied to the cellulosic fibers at a coat weight of from about 0.1 to about 25%, more particularly from about 5 to about 20% based upon dry fiber weight of the finished substrate.

The latex coating can be applied to the cellulose web on one or both sides of the web and can be applied as a full coating or in a pattern. Furthermore, the coating can be applied so as to penetrate through the web or to remain primarily on the surface of the sheet with only slight penetration through the sheet. Coating compositions and methods for producing latex-coated substrates useful in the present invention are disclosed in U.S. Pat. No. 4,000,237 to Roberts.

Nonlimiting examples of cellulosic fibers include those selected from the group consisting of wood pulp fibers, cotton fibers, hemp fibers, jute fibers, flax fibers, and mixtures thereof.

Substrates made from natural materials consist of webs or sheets most commonly formed on a fine wire screen from a liquid suspension of the fibers. See C. A. Hampel et al., The Encyclopedia of Chemistry, third edition, 1973, pp. 793-795 (1973); The Encyclopedia Americana, vol. 21, pp. 376-383 (1984); and G. A. Smook, Handbook of Pulp and Paper Technologies, Technical Association for the Pulp and Paper Industry (1986).

Latex coated cellulosic fibers suitable for use as the substrate are available from various sources. Examples of suitable latex coated cellulosic materials include, but are not limited to, a 56 g/m² paper with 15% latex binder manufactured by Duni Paper under the designation DL20195, a 65 g/m² cellulose EVA substrate and a 75 g/m² cellulose and EVA substrate available from Concert under the designations ME065.D0031 and STD75L, respectively, various Georgia Pacific substrates (Grade 314 (52 g/m²); Grade 316 (52 g/m²); and Grade 331 (56 g/m²)), and a 50 g/m² substrate manufactured by Kimberly Clark under the trade name VIVA.

Methods of making paper substrates, including apertured substrates, are well known in the art. Generally, these paper substrates can be made by air-laying or water-laying processes in which the fibers are first cut to desired lengths from long strands, passed into a water or air stream, and then deposited onto a screen or belt through which the fiber-laden air or water is passed. The resulting layer, regardless of its method of production or composition, is then subjected to at least one of several types of bonding operations to anchor the individual fibers together to form a self-sustaining web. In the present invention the layer can be prepared by latex bonding of the fibers. Moreover, the substrates used in the present invention can consist of a single layer or multiple layers. In addition, a multilayered substrate can include films and other nonfibrous materials.

The substrates used to form the personal cleansing articles of the present invention may contain apertures or openings in the cleansing surface of the substrate. Such apertures may be generally circular in shape or may be openings of other shapes, including squares, rectangles, trapezoids, diamonds, hexagons, irregular shapes and the like. Such apertures need not be uniform in size and shape, but preferably will be substantially uniform in both size and shape.

The apertures, when present, in the cleansing surface of the substrate will generally range in average diameter between about 0.5 mm and 5 mm. More preferably, the apertures will range in size between about 1 mm to 4 mm in average diameter. Preferably no more than about 10% of the apertures in the cleansing surface of the substrate will fall outside these size ranges. More preferably no more than about 5% of the apertures in the cleansing surface will fall outside these size ranges. For apertures which are not circular in shape, the “diameter” of the aperture refers to the diameter of a circular opening having the same surface area as the opening of the non-circular shaped aperture.

Within the cleansing surface of the substrate, the apertures will generally occur at a frequency of from about 0.5 to 12 apertures per straight linear centimeter. More preferably the apertures in the cleansing surface will occur at a frequency of from about 1.5 to 6 apertures per straight linear centimeter.

The apertures, when used, should at least be placed within the cleansing surface of the substrate element herein. Such apertures need not protrude completely through to the surface of the substrate which is opposite to the cleansing surface. When two or more plies or layers are used to form the water-insoluble substrate, apertures may or may not be placed in all of the plies or layers. Frequently, as noted more fully hereinafter, the substrate may comprise two layers one of which includes the cleansing surface and may be apertured. Not typically apertured is the other layer or ply which forms a backing for the substrate with the cleansing surface.

Apertures may be formed in the cleansing surface of the water-insoluble substrate as such a substrate, or layer thereof, is being formed or fabricated. Alternatively, apertures may be formed in the cleansing surface after the substrate, or ply or layer thereof, comprising the cleansing surface has been completely formed.

The substrate can be made into a wide variety of shapes and forms including flat pads, thick pads, thin sheets, ball-shaped implements, irregularly shaped implements, and having sizes ranging from providing a cleansing surface area of at least about 5 cm². The exact size will depend upon the desired use and product characteristics. Especially convenient are square, circular, rectangular, or oval pads having a cleansing surface area of from about 6 cm² to 1000 cm², preferably from about 65 cm² to about 775 cm², and more preferably from about 150 cm² to about 400 cm² and a thickness of from about 1 mil to about 500 mil, preferably from about 5 mil to about 250 mil, and more preferably from about 10 mil to about 100 mil.

Furthermore, it is desirable for the substrates of the present invention to have rounded corners. This feature prevents the tendency of water to accumulate at the comers of an unrounded rectangular, e.g., square, substrate. Corners, preferably all of the corners on the substrate, can be rounded to provide a radius of from about 1 to 4 cm. Preferably the rounded corners will have a radius of from about 2 to 3 cm.

The water insoluble substrates of the present invention can comprise one or more layers, each having different textures and abrasiveness. The differing textures can result from the use of different combinations of materials or from the use of different manufacturing processes or a combination thereof. A dual textured substrate can be made to provide the advantage of having a more abrasive side for exfoliation and a softer, absorbent side for gentle cleansing. In addition, separate layers of the substrate can be manufactured to have different colors, thereby helping the user to further distinguish the surfaces.

In accordance with one embodiment of the present invention, the water-insoluble substrate is one wherein the substrate includes at least two layers or plies. The top layer is extensible when it is wetted and, in accordance with this embodiment, is apertured. The base layer comprises latex-coated cellulosic fibers and is less wet extensible when wetted than the top layer. Selected portions of the top layer are joined to the base layer to inhibit wet extension of the top layer in the plane of the top layer. When the top layer is wetted, the base layer constrains extension of the top layer in the plane of the top layer. As a result, portions of the top layer deform, such as by buckling or puckering, in the Z-direction (perpendicular to the plane of the top layer).

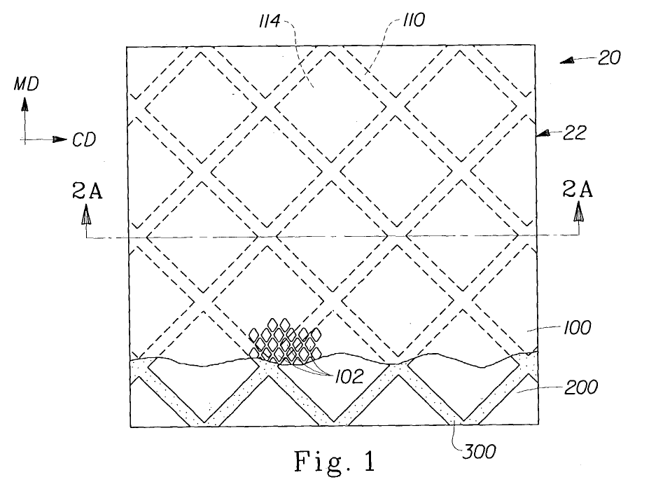

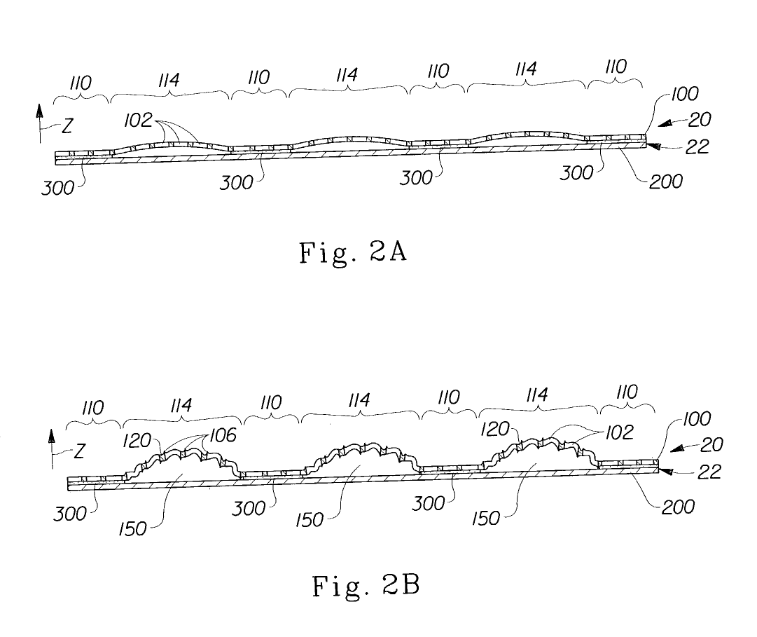

One embodiment of a water insoluble substrate having at least a portion that is wet extensible is illustrated in FIGS. 1-2 of the drawings. In this embodiment, the present invention comprises a multiple layer, i.e., two-ply, disposable wiping article 20. The disposable cleansing and conditioning article 20 comprises a substrate designated generally by reference numeral 22. The substrate 22 comprises a top layer 100 and a base layer 200. The top layer 100 is extensible, and in particular is extensible when wetted, e.g., the top layer is wet extensible. By “wet extensible” is meant that a material has a tendency to elongate in at least one direction when wetted. In general, “wetted” refers to wetting with aqueous solutions, such as water, which are capable of inducing extension in the top layer. For example, water relaxes the crepe in foreshortened paper, thereby causing an extension of the paper in at least one direction in the plane of the paper. Without being bound by theory, the relaxation of crepe may be a result of the loss of hydrogen bonds within the paper structure due to the presence of water. However, any fluid, mixture, or solution which could cause this crepe relaxation would be considered to “wet” the article. The base layer 200 is relatively less wet extensible when wetted than the top layer 100. Extensibility is measured according to the “Wet Extensibility Test” described below, and is reported as a percentage

Selected portions of the top layer 100 are joined, directly or indirectly, to base layer 200 to inhibit wet extension of the top layer in the plane of the top layer. In FIGS. 1 and 2, selected portions of the top layer 100 are joined to the base layer 200 to provide continuous bonded regions designated 110 and discrete unbonded regions 114.

In a preferred embodiment shown in FIG. 1, the bonded regions 110 are shown as a continuous network of intersecting lines forming generally diamond-shaped unbonded regions 114. The width and spacing of the intersecting lines of bonded regions 110, may be adjusted to the desired size and spacing of the diamond-shaped unbonded regions 114. The continuous network of intersecting lines may be virtually any pattern, resulting in unbonded regions of virtually limitless geometric shapes, including, for example, squares, rectangles, and triangles. The network need not be completely continuous, nor limited to a pattern of straight or uniform lines, but may, for example, be a network resulting in circular, oval, or other non-polygonal geometric shapes. An adhesive, such as a hot melt adhesive, designated by reference numeral 300 in FIGS. 1-2 can be used to join the top layer 100 to base layer 200.

When the top layer is wetted, there is a tendency for the top layer 100 to expand along one or more directions in the plane of the top layer. (The plane of the top layer is parallel to the plane of FIG. 1). However, because of the relatively lower wet extensibility of the base layer 200, the base layer constrains extension of the top layer 100 in the plane of the top layer. As a result, the unbonded regions 114 of the top layer 100 deform, such as by buckling or puckering in the Z-direction, perpendicular to the plane of the top layer 100.

FIG. 2A is a cross-sectional illustration of the cleansing and conditioning article 20 prior to wetting of the top layer 100. As shown in FIG. 2A, the wiping article is generally flat prior to wetting. FIG. 2B is a cross-sectional illustration similar to that of FIG. 2A, but showing the article 20 after wetting of the top layer 100. FIG. 2B shows out of plane deformation of the top layer 100 upon wetting of the top layer 100. The Z-direction is indicated in FIGS. 2A and 2B. The deformation of the wetted top layer 100 provides the article 20 with elevated ridges 120 which increase the wet texture, wet caliper (thickness) and wet bulk of the article 20. The elevated ridges 120 also provide pockets 150 disposed between the unbonded portions of the top layer 100 and the underlying portions of the base layer. In particular, the article 20 has a wet caliper to dry caliper ratio which is greater than 1.0, preferably at least about 1.1, more preferably at least about 1.2, and most preferably at least about 1.4. The wet caliper to dry caliper ratio is a measure of the thickness of the article 20, when wetted, relative to the thickness of the dry article 20 prior to wetting. The wet caliper to dry caliper ratio is measured according to the procedure “Wet Caliper to Dry Caliper Ratio” provided hereinafter.

In the preferred embodiment shown in FIG. 1, the top layer 100 is apertured, the top layer 100 comprising a plurality of apertures 102 which extend through the thickness of the top layer 100. Apertures add greatly to the desired texture and bulk of wiping article 20. In FIGS. 1-2, apertures 102 are shown on only a portion of the top layer 100 for clarity. When an apertured top layer is used, the deformation of the wetted top layer 100 again provides the article 100 with elevated ridges 120 which increase the wet texture, wet caliper (thickness) and wet bulk of the article 20. However, in this embodiment, the elevated ridges 120 have apertures 102 which provide a flow path through which liquids and/or small particles can enter the pockets 150.

Additionally, since the article 20, or alternate single ply apertured substrate, is used with, or includes a lathering agent, such as a surfactant, the apertures 102 can aid in the incorporation of air during the lathering process, thereby improving lather generation. For instance, a portion of the article 20 can be coated with or otherwise treated with a surfactant composition, as described more fully below. The article 20 can be wetted with water to activate the surfactant, and the airflow generated through the apertures 102 during use of the article (e.g. washing or wiping) can help to generate lather.

The size and number of the apertures 102 can influence the speed of lather generation and the quality of lather produced. A relatively small number of relatively large apertures 102 will tend to reduce the time required to generate lather, but will yield relatively large lather bubbles with a translucent appearance. On the other hand, a relatively larger number of relatively smaller apertures 102 will tend to reduce bubble size, thereby increasing lather creaminess and opacity, but at the expense of increasing the time required to generate lather.

Another advantage has been identified when top layer 100 is apertured. As shown in FIG. 2B, in addition to the formation of elevated ridges 120, the wet extension of top layer 100 around apertures 102 forms what can best be described as cusps 106, or surface irregularities formed by the apertures 102. Cusps 106 give added texture to the surface on the side of apertured surface 22 of top layer 100. This added texture may be modified as needed by adjusting the size and spacing of apertures 102.

In a preferred embodiment, a wipe 20 of the present invention comprises an apertured cellulosic paper top layer bonded to a latex-coated cellulosic in a continuous network of intersecting lines defining diamond-shaped unbonded regions. This combination of materials and bonding method and pattern provides for a preferred wipe that exhibits increased texture and bulk on one side upon wetting, while maintaining relatively smooth softness on the other side, and has a wet caliper greater than the dry caliper.

In addition to the above description, it has been found that an additional processing step involving heating the substrate after bonding can be used to further improve texture and bulk, as well as the general aesthetic qualities of the wipe. Without being bound by theory, it is believed that the process of heating causes the thermoplastic adhesive to contract, thereby further causing out-of-plane (Z-direction) deformation of the top layer, as well as the base layer. By contracting in the plane of the wipe article, both layers experience a Z-direction increase in caliper, giving increased overall caliper with a pleasing quilted look.

For example, a wipe that has been adhesively bonded with an EVA hot melt adhesive (one suitable adhesive is a hot melt adhesive commercially available as H1382-01 from Ato-Findley Adhesives of Wauwatosa, Wis.), may increase in caliper between 10-20% after a post-lamination heat treatment. In this case, a suitable hot melt adhesive is applied and the resulting article is cooled to room temperature. Heat treatment may then be performed, for example, raising the temperature to 100 degrees Celsius for 20 seconds is sufficient to initiate contraction of the polymer network. While not being bound by theory, it is believed that for this process to be effective, the pattern of bonding must be a continuous or essentially continuous network. Discrete bond sites may not sufficiently contract to improve the appearance of the article.

Top Layer

Referring to the components of the article 20 in more detail, suitable materials from which the top layer 100 can be formed include foreshortened (such as by creping) wetlaid paper webs. Other suitable materials can include woven materials, nonwoven materials, foams, battings, and the like.

The top layer 100 should be constructed to have a wet extensibility of at least 4 percent, more preferably at least about 10 percent, and still more preferably at least about 20 percent. In one embodiment, the top layer has a wet extensibility of at least about 25 percent. Preferably, the difference between the wet extensibility of the top layer and the wet extensibility of the base layer (the wet extensibility of the base layer subtracted from the wet extensibility of the top layer) is at least about 4 percent, more preferably at least about 10 percent, and still more preferably at least about 25 percent.

The fibers or filaments of the top layer 100 can be natural (e.g. cellulosic fibers such as wood pulp fibers, cotton linters, and bagasse fibers) or synthetic (e.g. polyolefins, rayon, polyamides or polyesters), or combinations thereof.

In another preferred embodiment, the top layer 100 comprises a wetlaid paper web of cellulosic wood pulp fibers which is foreshortened at least about 4 percent, more preferably at least about 10 percent, and still more preferably at least about 20 percent, by dry creping. The top layer may comprise crepe ridges corresponding to the foreshortening of the top layer 100. The machine direction (MD) and cross machine direction (CD) are indicated in FIG. 1. The machine direction corresponds to the direction of manufacture of the paper web of top layer 100. The crepe ridges are generally perpendicular to the machine direction, and generally parallel to the cross machine direction of the paper web of top layer 100.

The paper web of the top layer 100 can have a basis weight of between about 15 to about 65 grams per square meter. In a preferred embodiment, the basis weight of the top layer is between about 25 to about 45 grams per square meter, and in a more preferred embodiment, the basis weight of the top layer 100 is about 35 grams per square meter.

It is believed that the paper strength can significantly alter the overall appearance of the complete article. The amount of crepe input to the top layer is directly proportional to the amount of planar expansion and thereby the amount of caliper generated upon wetting. However, if the wet strength of the paper article is insufficient, the “buckles” may collapse to form a more “wrinkled” product having less caliper. Therefore both crepe and wet strength can be adjusted to provide an amount of texture based on the intended use of the article. Wet burst measurements can be made with a Thwing-Albert Burst Tester model number 1300-77, which tests peak load of a fully wetted substrate. The test utilizes a 1.3 cm ball diameter, a 12.7 cm/min ball velocity, and clamps the test sample around a 8.9 cm diameter circle perpendicular to the motion of the ball. Peak load wet burst strengths are between 100 and 1200 grams per ply. More preferably between 400 and 700 grams per ply and most preferably between 500 and 600 grams per ply.

In a more preferred embodiment, the top layer 100 comprises an apertured wetlaid paper web of cellulosic wood pulp fibers. The apertures 102 can be formed in the top layer 100 in any suitable manner. For instance, the apertures 102 can be formed in the top layer 100 during formation of the paper web of the top layer 100, or alternatively, after the paper web of the top layer 100 is manufactured. In one embodiment, the paper web of the top layer 100 is produced according to the teachings of one or more of the following U.S. patents: U.S. Pat. No. 5,245,025 issued Sep. 14, 1993 to Trokhan et al.; U.S. Pat. No. 5,277,761 issued Jan. 11, 1994 to Phan et al.; and U.S. Pat. No. 5,654,076 issued Aug. 5, 1997 to Trokhan et al. In particular, U.S. Pat. No. 5,277,761 at Column 10 discloses formation of a paper web having apertures.

Prior to wetting of the top layer, the creped top layer 100 can have between about 0.5 and 50 apertures per square centimeter, and more preferably between about 0.5 and 16 apertures per square centimeter. Wetting a creped paper web causes the web, if unrestrained, to expand in at least one direction, such as the machine direction, so that the number of apertures 102 per square area after wetting can be smaller than the number of apertures per square area prior to wetting. Similarly, when apertures are formed in a paper web, and the paper web is subsequently creped, the number of apertures per square area prior to creping will be smaller than the number of apertures per square area after creping. Accordingly references to paper web dimensions refer to dimensions after creping and prior to wetting.

The apertures 102 can comprise between about 15 and about 75 percent of the total surface of the top layer 100. The apertures 102 may be bilaterally staggered (staggered in both the machine and cross machine directions) in a repeating, nonrandom pattern. In one embodiment, the top layer 100 comprises a paper web which is dry creped 25 percent (25 percent foreshortening) with greater than about 25 percent wet extensibility, and has about 6 to 8 apertures, 102, per square centimeter, the apertures 102 having a length of about 0.25 to 0.46 centimeters and a width of about 0.17 to 0.38 centimeter, and a distance between apertures 106 of about 0.12 to to about 0.20 centimeter.

The paper web is manufactured by first forming an aqueous papermaking furnish. The furnish comprises papermaking fibers, and can further comprise various additives. U.S. Pat. No. 5,223,096 issued Jun. 29, 1993 to Phan et al. discloses various wood pulps and papermaking additives. A suitable paper web for making the top layer 100 can be manufactured according to the description provided in U.S. Pat. No. 6,060,149, to Nissing et al.

Base Layer

The top layer 100 is joined to the base layer 200 to constrain extension of selected portions of the top layer 100 when the top layer is wetted. The base layer 200 has a lower wet extensibility than that of the top layer 100.

In accordance with one aspect of the present invention, the base layer 200 comprises a web of latex-coated cellulosic fibers. The paper web of the base layer 200 can have a basis weight of between about 25 to about 150 grams per square meter. In accordance with a particular embodiment, the basis weight of the base layer is between about 40 to about 100 grams per square meter, and in a more particular embodiment, the basis weight of the base layer 200 is about 45 to about 75 grams per square meter.

Latex coated cellulosic fibers suitable for use as base layer 200 are available from various sources. Examples of suitable latex coated cellulosic materials include, but are not limited to, a 56 g/m² paper with 15% latex binder manufactured by Duni Paper under the designation DL20195, a 65 g/m² cellulose EVA substrate and a 75 g/m² cellulose and EVA substrate available from Concert under the designations ME065.D0031 and STD75L, respectively, various Georgia Pacific substrates (Grade 314 (52 g/m²); Grade 316 (52 g/m²); and Grade 331 (56 g/m²)), and a 50 g/m² substrate manufactured by Kimberly Clark under the trade name VIVA.

Ply Bonding

Selected portions of the top layer 100 can be joined directly (or indirectly such as through a third component) to the base layer 200 in a predetermined bonding pattern to provide a plurality of bonded and unbonded regions of the top layer 100. In FIGS. 1-2 the bonded regions are designated 110, and the unbonded regions arc designated 114. Each of the first and base layers 100 and 200 can have a machine direction, and the first and base layers can be bonded so that the machine direction of the top layer is generally parallel to the machine direction of the base layer.

The top layer 100 and the base layer 200 can be joined using any suitable method, including but not limited to adhesive bonding, mechanical bonding, thermal bonding, mechanical-thermal bonding, ultrasonic bonding, and combinations thereof. In particular, in a preferred embodiment, adhesive is applied by printing methods, such as gravure printing, reverse gravure printing, screen printing, flexographic printing, and the like. In one preferred embodiment, EVA hot melt adhesive may be screen printed in a lattice pattern generally as shown in FIG. 1. The suitable screen for this embodiment is a 40 mesh Galvano screen manufactured by Rothtec Engraving Corp., New Bedford, Mass.

The adhesive is preferably water insoluble so that the article 20 can be wetted with water without delamination of the first and base layers. The adhesive is preferably also surfactant tolerant. By “surfactant tolerant” it is meant that the bonding characteristics of the adhesive are not degraded by the presence of surfactants. Suitable adhesives include EVA (ethylene vinyl acetate) based hot melt adhesives. One suitable adhesive is a hot melt adhesive commercially available as H1382-01 from Ato-Findley Adhesives of Wauwatosa, Wis.

With reference to FIGS. 1 and 2, the hot melt adhesive can be applied to the nonwoven base layer 200 in a continuous network defining a discontinuous plurality of unbonded regions 114. In one preferred embodiment, as shown in FIG. 1, the adhesive is applied as parallel, spaced apart lines in a first direction, intersected by parallel, spaced apart lines in a second direction. The intersecting lines form diamond-shaped patterns of unbonded regions in the final wipe. In the embodiment shown in FIG. 1, the hot melt adhesive can be applied in lines having a width of about 0.025 centimeter to about 1.25 centimeter, preferably about 0.125 to about 0.18 centimeter. The spacing between adjacent lines of adhesive can be about 0.5 to 5.0 centimeter, preferably about 1.0 to 1.5 centimeter.

When applied as parallel stripes, lines, or bands, the adhesive can be applied to the base layer 200 using a slot coating applicator. A suitable slot coating applicator is a Nordson MX series hot melter with extrusion head commercially available from the Nordson Company of Norcross, Ga. The H1382-01 adhesive referenced above can be applied to the base layer 200 at a temperature of about 350° Fahrenheit, at an application level of about 0.03 grams of adhesive per square inch. Immediately following application of the adhesive to the base layer 200, the base layer 200 and the paper top layer 100 can be bonded together by pressing the two layers 100 and 200 together with the adhesive disposed between the base layer 200 and the top layer 100. One suitable means for pressing the two layers 100 and 200 together is by passing the two layers through a nip formed between two rollers, with the rollers loaded to provide adequate nip pressure for bonding.

The resulting laminate of the top and base layers can have an average dry caliper of about 28.5 mils (0.072 centimeter), an average wet caliper of about 32.1 mils (0.082 centimeter), and a wet caliper to dry caliper ratio of about 1.1. The dry caliper, wet caliper, and wet caliper to dry caliper ratio are measured as described below under “Wet Caliper to Dry Caliper Ratio.”

Wet Extensiblity Test

The wet extensibility of a layer, such as the layer 100 or the layer 200, is determined using the following procedure. Samples are conditioned at 70 degrees Fahrenheit and 50 percent relative humidity for two hours prior to testing.

First, the direction of greatest wet extensibility in the plane of the layer is determined. For dry creped paper webs, this direction will be parallel to the machine direction, and generally perpendicular to the crepe ridges.

If the direction of greatest wet extensibility is not known, the direction can be determined by cutting seven samples from a sheet with sample lengths oriented between 0 degrees and 90 degrees, inclusive, with respect to a reference line drawn on the sheet. The samples are then measured as set forth below to determine the direction of greatest wet extensibility.

Once the direction of the greatest wet extensibility is determined, 8 samples are cut to have a length of about 18 centimeters measured parallel to the direction of greatest wet extensibility, and a width of at least 2.54 centimeters. The samples are cut from unbonded portions of the layers 100 and 200, or, if unbonded portions having the above dimensions cannot be cut from the article 20, then samples are cut from the layers 100 and 200 prior to bonding the layers together. Two marks are placed on each sample, such as with an ink pen. The marks are spaced apart 12.7 centimeters as measured parallel to the direction of greatest wet extensibility. This 12.7 centimeter length is the initial dry test length of the sample.

Each sample is thoroughly wetted by submerging the sample in distilled water for 30 seconds in a water bath. Each sample is removed from the water bath and immediately supported to hang vertically so that a line through the two marks is generally vertical. The wet sample is supported such that the support does not interfere with extension between the two marks (e.g., with a clip which does not contact the sample between the two marks). The wet test length of the sample is the distance between the two marks. The distance is measured within 30 seconds of removing the sample from the water bath.

For each sample, the percent wet extension is calculated as

Sample Wet Extension=(wet test length−initial dry test length)/(initial dry test length)×100

For example, for a measured wet test length of 16.5 centimeters and an initial dry test length of 12.7 centimeters, the wet extension is ((16.5−12.7)/12.7)×100=30 percent. The wet extensibility of the samples is the average of 8 calculated values of sample wet extension.

In the preferred embodiment of the invention herein, the top layer preferably has a wet extensibility of at least about 4 percent, more preferably at least about 10 percent, and still more preferably at least about 20 percent as measured using the “Wet Extensibility Test” provided hereinbefore. The top layer can be foreshortened to provide the desired wet extensibility. In one embodiment, the top layer comprises a wet laid, apertured paper web which is foreshortened about 30 percent by dry creping. The base layer has a wet extensibility that is less than that of the top layer. The wet extensibility of the top layer minus the wet extensibility of the base layer is preferably at least about 4 percent, more preferably at least about 10 percent, and still more preferably at least about 20 percent.

Wet Caliper to Dry Caliper Ratio

The wet caliper to dry caliper ratio is measured using a Thwing-Albert Instrument Co. Electronic Thickness Tester Model II, using the following procedure. Samples are conditioned at 70 degrees Fahrenheit and 50 percent relative humidity for two hours prior to testing.

The dry caliper of the article 20 is measured using a confining pressure of 95 grams per square inch and a load foot having a diameter of 2 inches. The dry caliper is measured for eight samples. For each sample, the caliper is measured with the load foot centered on an unbonded region of the top layer 100. The eight caliper measurements are averaged to provide an average dry caliper.

Each sample is then wetted by submerging the sample in a distilled water bath for 30 seconds. The sample is then removed from the water bath and drained by hanging vertically for about five seconds. The caliper of the wet sample is measured within 30 seconds of removing the sample from the bath. The wet caliper is measured in the same location in which the dry caliper was previously measured. The eight wet caliper measurements are averaged to provide an average wet caliper. The wet caliper to dry caliper ratio is the average wet caliper divided by the average dry caliper.

The wet caliper to dry caliper ratio is the average wet caliper divided by the average dry caliper.

In the preferred two-ply substrate herein, the disposable cleansing and conditioning article can have a wet caliper to dry caliper ratio greater than 1.0, more preferably at least about 1.1, even more preferably at least about 1.2, and most preferably at least about 1.4.

Lathering Surfactant

Besides the water-insoluble substrate, the articles of the present invention also comprise one or more lathering surfactants which are releasably associated with the water-insoluble substrate. Thus the lathering surfactants can be added onto or impregnated into the substrate. Generally this will be done prior to the point of use of the article, i.e., the surfactants will be combined with the article and the article dried before the article is ultimately wetted for use. Preferred articles of the present invention comprise a sufficient amount of one or more lathering surfactants such that the articles are capable of generating ≧30 ml of Lather Volume (medium hardness water at 35° C. (95° F.)) according to the Lather Volume Test described below.

Generally the articles will contain from about 0.5% to 250%, by weight of the substrate, of a lathering surfactant that is releasably associated with the substrate. Preferably, the articles of the present invention comprise from about 0.5% to about 50%, more preferably from about 0.75% to about 30%, and most preferably from about 1% to about 20%, based on the weight of the water insoluble substrate, of a lathering surfactant component.

By a lathering surfactant is meant a surfactant, which when combined with water and mechanically agitated generates a foam or lather sufficient to cause the article, as a whole, to provide a lather. Preferably, these surfactants or combinations of surfactants should be mild, which means that these surfactants provide sufficient cleansing or detersive benefits but do not overly dry the skin or hair, and yet meet the lathering criteria described above.

A wide variety of lathering surfactants are useful herein and include those selected from the group consisting of anionic lathering surfactants, nonionic lather surfactants, amphoteric lathering surfactants, and mixtures thereof. Nonlimiting examples of lathering surfactants useful in the compositions of the present invention are disclosed in U.S. Pat. No. 6,280,757, to McAtee et al., issued Aug. 28, 2001. Generally, the lathering surfactants do not strongly interfere with deposition of any conditioning agents that are present, e.g., are fairly water soluble, and usually have an HLB value of above 10. Cationic surfactants can also be used as optional components, provided they do not negatively impact the overall lathering characteristics of the required lathering surfactants.

Anionic Lathering Surfactants

Nonlimiting examples of anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975.

A wide variety of anionic lathering surfactants are useful herein. Nonlimiting examples of anionic lathering surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853, cited above.

Other anionic materials include phosphates such as monoalkyl, dialkyl, and trialkylphosphate salts.

Nonlimiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein is ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

Nonionic Lathering Surfactants

Nonlimiting examples of nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992).

Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Nonlimiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide, and mixtures thereof.

Amphoteric Lathering Surfactants

The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992).

Nonlimiting examples of amphoteric or zwitterionic surfactants are those selected from the group consisting of betaines, sultaines, hydroxysultaines, alkyliminoacetates, imninodialkanoates, aminoalkanoates, and mixtures thereof.

Preferred lathering surfactants for use herein are the following, wherein the anionic lathering surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isetlionate, sodium cetyl sulfate, sodium lauroyl lactylate, triethanolamine lauroyl lactylate, and mixtures thereof, wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

Lather Volume Test

The articles of the present invention preferably comprise enough of the lathering surfactant such that the articles are capable of generating greater than or equal to about 30 ml, more preferably greater than or equal to about 50 ml, even more preferably greater than or equal to about 75 ml, and most preferably greater than or equal to about 150 ml of Average Lather Volume. The Average Lather Volume is a measurement determined by the Lather Volume Test. This test provides a consistent measurement of the volume of lather/foam generated by the articles described herein. The Lather Volume Test protocol is described as follows:

(1) Hands are washed with Ivory bar before conducting the test. This step removes any soils which may affect the accuracy of the measurement.

(2) The test article is held open in the non-dominant hand with the edges turned up.

(3) 10 ml. of water (medium hardness of about 8-10 grains per gallon) at 35° C. (95° F.) is added onto the test article via a 10 cc syringe or a Brinkmann repipetter.

(4) The lather is then generated by rubbing the test article with the dominant hand in a circular motion between the palms for 6 seconds (˜2 rotations per second), using moderate pressure (e.g., 4 oz.), and allowing the article to ball-up between the palms of the hand.

(5) The test article is then held open in the non-dominant hand and an additional 10 ml of water (medium hardness of about 8-10 grains per gallon) at 35° C. (95° F.) is added onto the test article via a 10 cc syringe or a Brinkmann repipetter. The wetted article is again rubbed with the dominant hand (3 rotations) using moderate force (e.g, 4 oz.) so that the test article becomes balled-up between the palms.

(6) The test article is then opened and rubbed 5 times by holding one edge of the article in one hand and rotating the hand holding the other side to further activate lather.

(7) The test article is then flipped over and Step #6 is repeated using the other hand.

(8) The lather is gathered by holding the test article in a cupped hand and scraping the lather off the test article with the other hand, being careful to only scrape lather from the test article. The lather from the test article is placed into a graduated cylinder or beaker big enough to hold the generated lather. This procedure is repeated 5 times on the same test article, and the lather from each iteration is accumulated in the same graduated cylinder or beaker. The total accumulated lather from these iterations is designated as the Lather Volume.

(9) To achieve consistent results, the Average Lather Volume is reported as the average of three test sample replications of Steps 1-8.

Conditioning Component

The articles of the present invention will preferably further comprise a conditioning component which is useful for providing a conditioning benefit to the skin or hair during the use of the article. The conditioning component can comprise from about 0.05% to about 99%, preferably from about 0.1% to about 50%, and more preferably from about 1% to about 25% by weight of said water insoluble substrate.

The conditioning component useful in the present invention can comprise: a water soluble conditioning agent; an oil soluble conditioning agent; a conditioning emulsion; or any combination or permutation of the three. The oil soluble conditioning agent is selected from one or more oil soluble conditioning agents such that the weighted arithmetic mean solubility parameter of the oil soluble conditioning agent is less than or equal to 10.5. The water soluble conditioning agent is selected from one or more water soluble conditioning agents such that the weighted arithmetic mean solubility parameter of the water soluble conditioning agent is greater than 10.5. It is recognized, based on this mathematical definition of solubility parameters, that it is possible, for example, to achieve the required weighted arithmetic mean solubility parameter, i.e., less than or equal to 10.5, for an oil soluble conditioning agent comprising two or more compounds if one of the compounds has an individual solubility parameter greater than 10.5. Conversely, it is possible to achieve the appropriate weighted arithmetic mean solubility parameter, i.e., greater than 10.5, for a water soluble conditioning agent comprising two or more compounds if one of the compounds has an individual solubility parameter less than or equal to 10.5.

Solubility parameters are well known to the formulation chemist of ordinary skill in the art and are routinely used as a guide for determining compatibilities and solubilities of materials in the formulation process.

Nonlimiting examples of conditioning agents useful as conditioning agents include those selected from the group consisting of fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters and mixtures thereof.

More particularly, the conditioning agent may be selected from the group consisting of paraffin, mineral oil, petrolatum, stearyl alcohol, cetyl alchohol, cetearyl alcohol, behenyl alcohol, C10-30 polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, carnauba wax, beeswax, and silicone waxes.

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p.415-417 (1993).

Petrolatum, which is also known as petroleum jelly, is a colloidal system of nonstraight-chain solid hydrocarbons and high-boiling liquid hydrocarbons, in which most of the liquid hydrocarbons are held inside the micelles. See The Merck Index, Tenth Edition, Entry 7047, p. 1033 (1983); Schindler, Drug. Cosmet. Ind., 89, 36-37, 76, 78-80, 82 (1961); and International Cosmetic Ingredient Dictionary, Fifth Edition, vol. 1, p. 537 (1993).

Nonvolatile silicones such as polydialkylsiloxanes, polydiarylsiloxanes, and polyalkarylsiloxanes are also useful oils. These silicones are disclosed in U.S. Pat. No. 5,069,897, to Orr, issued Dec. 3, 1991.

The conditioning component preferably used in the present invention may also comprise a conditioning emulsion which is useful for providing a conditioning benefit to the skin or hair during the use of the article. The term “conditioning emulsion” as used herein means the combination of an internal phase comprising a water soluble conditioning agent that is enveloped by an external phase comprising an oil soluble agent. In preferred embodiments, the conditioning emulsion would further comprise an emulsifier. The conditioning emulsion comprises from about 0.25% to about 150%, preferably from about 0.5% to about 100%, and more preferably from about 1% to about 50% by weight of said water insoluble substrate. By a conditioning emulsion is meant a combination of an internal phase comprising a water soluble conditioning agent that is enveloped by an external phase comprising an oil soluble agent. In preferred embodiments, the conditioning emulsion would further comprise an emulsifier.

The conditioning emulsion comprises (i) an internal phase comprising water soluble conditioning agents as described above, and (ii) an external phase comprising oil soluble agents as described hereinbefore in the oil soluble conditioning agent section or in the “Materials Used to Increase Lipid Hardness Value” section in U.S. Pat. No. 6,153,208. In further embodiments, the conditioning emulsion further comprises an emulsifier capable of forming an emulsion of said internal and external phases. Although an emulsifier capable of forming an emulsion of the internal and external phases is preferred in the present invention, it is recognized in the art of skin care formulations that a water soluble conditioning agent can be enveloped by an oil soluble agent without an emulsifier. As long as the water soluble conditioning agent is enveloped by the oil soluble agent, thereby protected from being rinsed away during the cleansing process, the composition would be within the scope of the present invention.

Preferred embodiments of the present invention which contain conditioning emulsions comprise an emulsifier capable of forming an emulsion of the internal and external phases. In the emulsions of the present invention, the emulsifier is included in an effective amount. What constitutes an “effective amount” will depend on a number of factors including the respective amounts of the oil soluble agents, the type of emulsifier used, the level of impurities present in the emulsifier, and like factors. Typically, the emulsifier comprises from about 0.1% to about 20%, preferably from about 1% to about 10%, and more preferably from about 3% to about 6% by weight of the conditioning emulsion.

The emulsifiers useful in the present invention typically are oil soluble or miscible with the oil soluble external phase materials, especially at the temperature at which the lipid material melts. It also should have a relatively low HLB value. Emulsifiers suitable for use in the present invention have HLB values typically in the range of from about 1 to about 7 and can include mixtures of different emulsifiers. Preferably, these emulsifiers will have HLB values from about 1.5 to about 6, and more preferably from about 2 to about 5.

Weight Ratios and Weight Percentages

In the present invention, the weight ratio of the lathering surfactant to the conditioning component is preferably less than about 40:7, more preferably less than about 5:1, even more preferably less than about 2.5:1, and most preferably less than about 1:1.

In certain preferred embodiments of the present invention, the cleansing and conditioning component, which is defined as comprising a lathering surfactant and a conditioning component further comprising an oil soluble conditioning agent and a water soluble conditioning agent, the lathering surfactant comprises from about 1% to about 75%, preferably from about 10% to about 65%, and more preferably from about 15% to about 45%, by weight of the cleansing and conditioning component, and the conditioning component comprises from about 15% to about 99%, preferably from about 20% to about 75%, and more preferably from about 25% to about 55%, by weight of the cleansing and conditioning component.

Additional Ingredients

The compositions which are added onto or impregnated into the articles of the present invention may comprise a wide range of optional ingredients. Particularly useful are added polymers, various active ingredients, and cationic surfactants useful for delivering various non-conditioning or non-cleansing benefits of the skin or hair during the cleansing and conditioning process. Additional ingredients of these types are described in greater detail in Procter & Gamble; PCT Application No. WO 99/13861; published Mar. 25, 1999 (P&G Case 6840).

Other Optional Ingredients

The articles of the present invention can comprise a wide range of other optional components. These additional components should be pharmaceutically acceptable. The CTFA Cosmetic Ingredient Handbook, Second Edition, 1992, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference. Examples of these and other functional classes include: abrasives, absorbents, anticaking agents, antioxidants, vitamins, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, preservatives, propellants, reducing agents, skin bleaching agents, and sunscreening agents.

Also useful herein are aesthetic components such as fragrances, pigments, colorings, essential oils, skin sensates, astringents, skin soothing agents, and skin healing agents.

Methods of Manufacture

The disposable, single use personal care cleansing articles of the present invention can be manufactured by separately or simultaneously adding onto or impregnating into a water-insoluble substrate a lathering surfactant and optionally a conditioning component. If necessary, the resulting article can then be dried. By “separately” is meant that the surfactants and conditioning agents can be added sequentially, in any order without first being combined together. By “simultaneously” is meant that the surfactants and conditioning agents can be added at the same time, with or without first being combined together.

The lathering surfactant and/or the conditioning component can be added onto or impregnated into the substrate using any means known to those skilled in the art. These components can be applied using various spraying, soaking, coating or dipping techniques. Excess surfactant and/or conditioning component should be removed (e.g., by a nipping process). Thereafter, the treated substrate should be dried by conventional means.

For the described embodiment containing two layers, the lathering surfactant and/or the conditioning component can also be added onto or impregnated into either layer (100 or 200) in any sequence. Alternatively, the lathering surfactant and/or the conditioning component can be added onto or impregnated into the resulting combination of the top layer 100 and the base layer 200. Treatment with the lathering surfactant and/or the conditioning component can be achieved at anytime before or after joining the top layer 100 and the base layer 200. Despite the order of treatment, excess surfactant and/or conditioning component should be removed (e.g., by a nipping process). Thereafter, the treated material (e.g., the top layer 100, the base layer 200, both layers 100 and 200, or joined substrate) should be dried by conventional means.

For example, prior to joining the top layer 100 to the base layer 200, the base layer can be treated with the lathering surfactant. After joining the two layers, either of the outside surfaces (e.g., the unjoined surfaces) of layers 100 and/or 200 can be treated with the conditioning component. Alternatively, the lathering surfactants and conditioning agents can be added onto or impregnated into the base layer 200 at the same time prior to joining the two layers. Alternatively, the lathering surfactants and the conditioning agents can be combined together before adding onto or impregnating into the base layer 200.

Alternatively, prior to joining the two layers, the top layer 100 can be treated with the lathering surfactant employing methods which do not cause the top layer to elongate or extend. This can be achieved in the manufacturing of the top layer or by various application methods well known to those of ordinary skill in the art. Nonlimiting examples of application methods include extrusion coating and slot coating.

The surfactant, conditioning agents, and any optional ingredients can be added onto or impregnated into either layer (100 or 200) or the resulting joined layers (100 and 200) by any means known to those skilled in the art: for example, by spraying, jet printing, splashing, dipping, soaking, or coating (e.g., slot coating, gravure, etc.).

When water or moisture is used or present in the manufacturing process, the resulting treated substrate is then preferably dried so that it is substantially free of water. The treated substrate can be dried by any means known to those skilled in the art. Nonlimiting examples of known drying means include the use of convection ovens, radiant heat sources, microwave ovens, forced air ovens, and heated rollers or cans. Drying also includes air drying without the addition of heat energy, other than that present in the ambient environment. Also, a combination of various drying methods can be used.

Preferably, upon wetting with water during use, the articles of the present invention are capable of generating an Average Lather Volume of greater than or equal to about 30 ml, more preferably greater than or equal to about 50 ml, even more preferably greater than or equal to about 75 ml, and most preferably greater than or equal to about 150 ml.

Methods of Cleansing and Conditioning the Skin or Hair

The present invention also relates to a method of cleansing and conditioning the skin or hair with a personal cleansing article of the present invention. These methods comprise the steps of wetting with water a substantially dry, disposable, single use personal cleansing article comprising a water insoluble substrate, a lathering surfactant, and optionally a conditioning component, and contacting the skin or hair with such wetted article. In further embodiments, the present invention is also useful for delivering various active ingredients to the skin or hair.

The articles of the present invention are intended to be wetted with water prior to use. The article is wetted by immersion in water or by placing it under a stream of water. Lather is generated from the article by mechanically agitating and/or deforming the article either prior to or during contact of the article with the skin or hair. Preferably, upon wetting, the articles of the present invention generate an Average Lather Volume of greater than or equal to about 30 ml, more preferably greater than or equal to about 50 ml, even more preferably greater than or equal to about 75 ml, and most preferably greater than or equal to about 150 ml. The resulting lather is useful for cleansing and conditioning the skin or hair. During the cleansing process and subsequent rinsing with water, the conditioning agents and active ingredients are deposited onto the skin or hair. Deposition of conditioning agents and active ingredients are enhanced by the physical contact of the substrate with the skin or hair.

Without being limited by theory it is believed that the substrate significantly contributes to generation of lather and deposition of conditioning agents and any other active ingredients. It is believed that this increase in lathering and deposition is the result of the surface action of the substrate. As a result, milder and significantly lower amounts of surfactants may be employed. The decreased amount of required surfactant is believed to relate to the decrease in the drying effect of the skin or hair by the surfactants. Furthermore, the diminished amount of surfactant dramatically lowers the inhibitory action (e.g., via emulsification or direct removal by the surfactants) which surfactants exhibit regarding deposition of conditioning agents.

Further without being limited by theory, it is believed that the substrate also enhances deposition of conditioning agents and active ingredients. Since the invention is in dry form, the invention does not require emulsifiers, which can inhibit deposition of conditioning agents and active ingredients. Furthermore, because the skin conditioners and active ingredients are dried onto or impregnated into the substrate, they are transferred directly to the skin or hair by surface contact of the wetted article to the skin.

The substrate also enhances cleansing. The substrate can have differing textures on each side, e.g. a rough side and a smooth side. The substrate, especially the multilayered construction, acts as an efficient lathering and exfoliating implement. By physically coming into contact with the skin or hair, the substrate significantly aids in cleansing and removal of dirt, makeup, dead skin, and other debris.

Finally, a substrate having at least a portion that is wet extensible provides the desired qualities (e.g., proper texture, thickness, and bulk) of a washcloth.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable, single use personal cleansing article especially useful for cleansing skin or hair, which article comprises:
(A) a water-insoluble substrate having at least one cleansing surface; and
(B) from about 0.5% to about 250% by weight of said substrate of a lathering surfactant releasably associated with said substrate;
wherein said water-insoluble substrate comprises a top layer and a base layer, said base layer comprising latex-coated cellulosic fibers.

2. An article according to claim 1 wherein said lathering surfactant comprises from about 0.5% to about 50% by weight of said water insoluble substrate.

3. An article according to claim 2 which is substantially dry prior to use.

4. An article according to claim 1 wherein said latex-coated cellulosic fibers comprise at least one material selected from the group consisting of acrylic, polyvinyl alcohol (PVA), vinyl acrylic, acrylic multipolymer, ethylene vinyl acetate (EVA) and copolymers thereof.

5. An article according to claim 4 wherein said latex-coated cellulosic fibers comprise from about 0.1% to about 25% latex coating based upon dry fiber weight of the finished substrate.

6. An article according to claim 1 wherein the cleansing surface of said substrate contains a plurality of apertures which range in average size from about 0.5 to 5 mm in diameter and which are located within said cleansing surface of said substrate at a frequency of from about 0.5 to about 12 per linear centimeter.

7. An article according to claim 6 wherein the average size of said apertures ranges from about 1 mm to about 4 mm and the frequency of the apertures in the substrate ranges from about 1 to 6 per linear centimeter.

8. An article according to claim 1 further comprising a conditioning component added onto or impregnated into said substrate.

9. An article according to claim 8 wherein said conditioning component comprises at least one material selected from the group consisting of fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters and mixtures thereof.

10. An article according to claim 9 wherein said conditioning component comprises at least one material selected from the group consisting of paraffin, mineral oil, petrolatum, stearyl alcohol, cetyl alchohol, cetearyl alcohol, behenyl alcohol, C10-30 polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, camauba wax, beeswax, and silicone waxes.

11. An article according to claim 1 wherein said lathering surfactant is selected from the group consisting of anionic lathering surfactants, nonionic lathering surfactants, amphoteric lathering surfactants, and mixtures thereof.

12. An article according to claim 11 wherein said anionic lathering surfactant is selected from the group consisting of sarcosinates, sulfates, isethionates, phosphates, taurates, lactylates, glutamates and mixtures thereof; wherein said nonionic lathering surfactant is selected from the group consisting of amine oxides, alkyl glucosides, alkyl polyglucosides, polyhydroxy fatty acid amides, alkoxylated fatty acid esters, sucrose esters, and mixtures thereof; and wherein said amphoteric lathering surfactant is selected from the group consisting of betaines, sultaines, hydroxysultaines, alkyliminoacetates, iminodialkanoates, aminoalkanoates, and mixtures thereof.

13. An article according to claim I wherein said water-insoluble substrate comprises:
(A) a top layer, the top layer being wet extensible in the plane of the top layer when the top layer is wetted; and
(B) a base layer which is less wet extensible when wetted than is said top layer; wherein selected portions of said top layer are joined to said base layer in a manner which is sufficient to inhibit wet extension of said top layer in the plane of said top layer.

14. The article of claim 13 wherein said top layer is apertured and has a wet extensibility of at least about 4 percent.

15. The article of claim 14 wherein said top layer comprises a creped paper web and said base layer is non-apertured and comprises said latex-coated cellulosic fibers.

16. The article of claim 15 wherein selected portions of said top substrate layer are adhesively bonded to said base substrate layer to provide a plurality of generally parallel, spaced apart bonded regions and a plurality of generally parallel, spaced apart unbonded regions within said substrate.

17. The article of claim 16 wherein selected portions of said top substrate layer are adhesively bonded to said base substrate layer to provide a continuous network bonded region which defines a plurality of discrete unbonded regions.

18. An article according to claim 1 wherein the article is capable of generating an Average Lather Volume of greater than or equal to about 30 ml upon wetting.

19. An article according to claim 18 wherein the article is capable of generating an Average Lather Volume of greater than or equal to about 75 ml upon wetting.

20. An article according to claim 1 wherein the fibers are formed into a sheet, mat, or pad layer.

21. An article according to claim I wherein said substrate is made by air-laying or water-laying processes in which the cellulosic fibers are cut to desired lengths, passed into a water or air stream, and then deposited onto a screen or belt through which the fiber-laden air or water is passed.

22. An article according to claim 1, wherein said substrate has rounded corners, said corner having a radius of from about 2 to 3 cm.

23. A method of manufacturing a disposable, single use personal care cleansing article comprising the step of adding at least one lathering surfactant onto or impregnating at least one lathering surfactant into a water-insoluble substrate having a cleansing surface wherein said substrate comprises latex-coated cellulosic fibers; and wherein said resulting article is substantially dry.

24. A method according to claim 23 wherein said substrate contains apertures ranging in average diameter from about 0.5 mm to about 5 mm and having a frequency of from about 0.5 to about 12 apertures per linear centimeter.

25. A method of cleansing the skin or hair with a personal cleansing article, comprising the steps of:
(A) wetting with water a substantially dry, disposable, single use personal cleansing article comprising:
(i) a water insoluble substrate having a cleansing surface, said substrate comprising latex-coated cellulosic fibers; and
(ii) at least one lathering surfactant, and
(B) contacting the skin or hair with said wetted article.

26. A method according to claim 25 wherein said article further comprises a conditioning agent for skin or hair.
